(19) United States
(12) Patent Application Publication
(10) Pub. No.: US 2003/0118668 A1
(43) Pub. Date: Jun. 26, 2003

(54) Aquamarine salts based fungicide

(76) Inventor: Gregory B. Yoos, San Diego, CA (US)

Correspondence Address: The law Offices of Joseph Chalverus, Lake City Professional Center, 2611 N. E. 125th Street, Seattle, WA, 98125, US

(21) Appl. No.: 10/028,044

(22) Filed: Dec. 20, 2001

(51) Int. Cl. A61K 33/14; A61K 33/00

(52) U.S. Cl. 424/680; 424/717

(57) ABSTRACT

A fungicide for the treatment of skin aliments prepared by mixing three parts aquamarine salts having a chloride to sodium ratio of 1.4:1, two parts ionized sodium chloride and one part sodium bicarbonate by weight. The dry mixture is blended then dissolved in water in the amount of four table spoons of mixture to one liter of water as a soaking bath for affected area of skin and nails. The mixture can also be dissolved to form a vaginal bath by dissolving two tablespoons of mixture into 16 ounces of water. The mixture may be ground into a fine powder and added to equal amounts by weight of petroleum gel to form a salve that is applied to affected areas for prolonged application. Food color may be added to provide aesthetic qualities to the mixture and resulting bath and douche.

### II. BACKGROUND OF THE INVENTION

1. Field of the Invention

This invention relates to materials and methods of treatment of persons and animals afflicted with skin infections. More particularly, this invention relates to the treatment of fungus based skin infections such as athlete's foot, nail and skin fungus infections, fungus infections of the feet and hands, softening of calluses, temporary relief of the discomfort of ingrown toenails, eczema, ringworm, dandruff, vaginal and other yeast infections and the like. Even more particularly, my invention relates to the use of commonly available products that when mixed together have a surprisingly beneficial effect for the persons and animals suffering from common fungal infections of skin, hair and nails areas and the consequences of these conditions, such as hardening of the tissues, formation of scales, flakes, dandruff and calluses.

2. Description of the Related Art

There are many known materials used to treat common skin problems. Many of these materials use skin conditioners, antibacterial and antifungal compounds which are expensive and often classified as controlled substances requiring a prescription to obtain and use. While many of these materials have shown a degree of success, their effectiveness is often outweighed by their costs and availability and in some cases toxicity to major internal organs and other side effects. It is known that sea water has been proven effective in treatment of common skin rashes and the like. The closest art to my invention involves the use of common, concentrated or modified sea-water to treat skin afflictions. The following U.S. patents are known to the discuss the beneficial use of sea-water to treat skin hair and nail problems, including fungus infections:

| PATENT NUMBER | ISSUE DATE | INVENTOR |
|---|---|---|
| 711,263 | Oct. 14, 1903 | Robertson |
| 2,223,142 | Nov. 26, 1940 | Weirich |
| 2,299,604 | Oct. 20, 1942 | Weirich |
| 4,342,743 | Aug. 3, 1982 | Panton-Moore |
| 4,581,226 | Apr. 8, 1986 | Dillon |
| 5,084,281 | Jan. 28, 1992 | Dillon |
| 5,492,693 | Feb. 20, 1996 | Miller, Jr. |

U.S. Pat. No. 711,263 to Roberton discusses the process of making a material, starting from sea-water, concentrating it removing some of the ingredients and then adding and adding acetic acid, the resulting material being useful for treatment of the diseases of the skin, scalp.

U.S. Pat. No. 4,342,743 to Panton-Moore relates to a preparation for the care and conditioning of the feet of land and sea-salts mixed with a papaya enzyme and salicylic acid, mixed to form a power to treat the feet.

Both U.S. Pat. Nos. 2,223,142 and 2,299,604 to Weirich relate to the treatment of athlete's foot using a solution having inorganic salts, and in particular sodium chloride from sea water, in combination with para-nitophenol or noxyacetic acid.

Both U.S. Pat. Nos. 4,581,226 and 5,084,281 to Dillon relate to the specific methods to process sea-water to produce a material useful for cleaning and treating common animal tissue.

U.S. Pat. No. 5,492,693 to Miller describes the use of commercial grade sea-salt made from evaporation of sea-water, the sea-salt is then reconstituted with water and combined with a skin conditioner for the treatment of animals suffering from various conditions.

It will be appreciated that these mixtures are often complicated and some need special equipment to make or use and require materials not readily available to the common person. In addition, commercial grade sea-salt made from evaporation of sea-salt is not readily available to everyone and when obtained may be contaminated with materials that may not be suitable for contact with animals or humans.

### III. BRIEF SUMMARY OF THE INVENTION

The present invention is based upon the inventor's observations that the use of the commonly available aquamarine salt mixtures manufactured for salt water aquariums for salt water fish combined with a mixture with iodized sodium chloride and sodium bicarbonate USP, then dissolved with tap water provides beneficial effects for all skin, hair and nail infections. Experimentation with this product proved beneficial for use to treat tinea cruris, persistent diabetic lesions, perianal rash as well as nail fungus on race horses. Given the range of effectiveness, it appears that the product provides general antifungal effects on a wide class of fungi. Aquamarine salt mixtures manufactured for salt-water aquariums are clean and do not contain the contaminants of common evaporated salt-water. The sodium chloride used in this invention is the ordinary salt commonly known as table salt used for consumption. The sodium bicarbonate used in this invention is the ordinary sodium bicarbonate used in conjunction with teeth brushing and consumed as an aid to indigestion. Ordinary tap water was proved to be just as effective as expensive distilled water or demineralized water. My combination of solids may be ground together into a fine powder then mixed with ointments to assist in application and retention on the body to achieve beneficial effects.

Accordingly, it is a general object of the present invention to provide a simple and effective means to treat fungus infections.

More specifically, it is an object of the present invention to provide an inexpensive treatment made from commonly available products to treat common skin aliments as described above.

It is also an object of this invention to teach a means for people to prepare their own antifungal solutions and ointments to self-treat skin infections and lesions without having to resort to time-consuming, expensive and in some cases highly toxic medical treatments.

Further objects and advantages of my invention will become apparent from a consideration of the drawings and ensuing description thereof.

### IV. DETAILED DESCRIPTION OF THE INVENTION

This invention uses commonly available aquamarine salt mixtures, used principally for aquariums and maintaining sea animals in captivity, that is mixed with ordinary iodized table salt and ordinary sodium bicarbonate USP then combined with water or ground into a fine powder to be mixed with ointments as described herein. The resulting compound proves beneficial for the treatment of common fungi that cause skin and nail disorders, such as nail and skin fungi of the feet (athlete's feet), infections of the hands and infections elsewhere such as vaginal yeast infections. My composition has proved effective for the treatment of other ailments such as softening of calluses on hands and feet shrinking and relief of the discomfort for hemorrhoids and provides temporary relief to the discomfort of ingrown toenails. It is believed safe and may be used prophylactically as a daily foot bath with the beneficial effects of relaxing the feet and body in general.

Aquamarine salts mixtures are available commercially in convenient amounts from any good pet store or store specializing in pet fish, particularly salt-water fish. This product is sold under many trademarks, such as CORALIFE and MARINEMIX. For the purposes of this disclosure, these mixtures are called aquamarine salts.

It is important as later described to distinguish aquamarine salts from sea salts, the salty solids resulting from evaporation of sea water. Chemically, aquamarine mixtures are not the same as residue derived from the simple evaporation of sea-water. Aquamarine salts when removed from the container have the appearance of a grayish-white, chunky granular material. Aquamarine salts are mixed with fresh water according to the specific instructions on the product label. Often warm water is used to assist in the dissolution of the aquamarine salts in the water. The resulting solution of aquamarine salts and water provides a swimming medium for captive salt-water fish. The solution contains all the needed salts and minerals required to maintain salt-water fish healthy in an artificial environment as opposed to the sea. With time, fresh water is used to replace any water evaporated from the solution.

The list of ingredients written on the MARINEMIX and CORALIFE container labels describe aquamarine salts to contain: chloride, magnesium, sodium, sulfur, potassium, calcium, bicarbonate, bromide, strontium, carbonate, fluoride, lithium, aluminum, rubidium, iodide, molybdate, zinc, vanadium, manganese, tin cobalt, barium, nickel, chromium, selenium with trace elements beneficial to marine life.

In 1865 Johann Georg Forchammer found that the relative ratio of chloride to sodium ions in sea water, taken from various parts of the world is always a constant 1.8:1. This is now known as Forchammer's principle or the Principle of Constant Proportions. This principle is accepted throughout in the marine industry. If the relative proportions of chloride to sodium in a solution differs from the Forchammer constant, then a of sample water is determined in the marine industry not to be from the sea. It is known that sea salts made from evaporation maintain their relative ratio of chloride to sodium of 1.8:1, that is 1.8 parts to one part.

Careful chemical analysis of the resulting mixture of my invention shows that aquamarine salts have a chloride to sodium ratio of 1.4:1, a ratio confirming that my invention has only one-half half of the relative proportion of chloride to sodium than the relative proportion of these elements found in sea water.

The sodium chloride used in my invention is ordinary ionized salt commonly known as table salt. The sodium bicarbonate used in this invention is ordinary sodium bicarbonate often used in conjunction with teeth brushing and consumed as an aid to indigestion. Ordinary tap water was proved to be just as effective as expensive distilled water or demineralized water as a solvent.

It should be clear that my invention is not the same as the mixture and combinations of aquamarine salts used in a marine aquarium. Chemical analysis of the solution of aquamarine salts mixed according to the instructions shows that aquamarine salts mixed with iodized sodium chloride and iodized sodium bicarbonate USP are not the resulting mixture produced from simple evaporation of salt water. There is a marked difference in the acidity of my compound as measured by the pH as compared to seawater. Additionally, the is a difference in specific gravity of my compound compared to the specific gravity of sea water or reconstituted artificial seawater prepared by the dissolution of aquamarine salts alone in water. Lastly, fish can not live in my described mixture of aquamarine salts with sodium chloride and bicarbonate.

While a theory of disinfection believes that disinfection results from the creation of a disfavorable environment to the pathogens, perhaps destroying their cellular wall, the principle of disinfection is not fully understood. Nevertheless, ionized sodium chloride and sodium bicarbonate salts mixed in my proportions have proven extremely effective for treatment of all sorts of skin and nail problems, including fungal infections. The mixture may be applied directly onto the skin, or in a solution with water, such as for a bath or mixed with a topical jelly, such as Petrolatum USP, to maintain contact with the skin for longer periods of time than by soaking in a bath.

The mixture once formulated, may be ground into a fine power and edible food coloring such as FD&C blue or green may be added to enhance the aesthetic appearance of the resulting mixture and to monitor the rate of dissolution of my mixture into solution. MIXTURE PROPORTIONS.

As described above, the mixture in a water solution once prepared may be used in a variety of ways such as a foot bath, or as a vaginal douche. The mixture may also be compounded with a petroleum gel or Petrolatum to form an ointment instead of dissolution into a solvent. The proportions of ingredients described below are only suggestive and preferred; they can be modified, that is the mixture ratios can be modified if the strength of the solution needs to be increased or decreased, according to the user, skin sensitivity and the like.

My invention comprises the preferred mixture of three parts of commercial grade aquamarine salts, combined with two parts of iodized sodium chloride and one part of sodium bicarbonate USP, by volume. The preferred order of mixing is to mix sodium bicarbonate into the aquamarine salts, then add the iodized sodium chloride and mix into a homogeneous compound. The compound may be ground into a fine power and food coloring may be added. I found that the amount of food grade coloring to be added depends upon the color and tint of the final product desired.

The grinding into a fine powder may be preferable done by a ball-mill commonly used to reduce dry compounds into a fine power or other similar means. While caking after the compounding is normal and will not appreciably affect the potency of my invention, it is preferred that the compounding be performed under conditions where the humidity does not affect the ingredients or resulting product.

For maximum treatment of skin, fingernail and toenails tissue, up to a ratio of four tablespoons of my compound is dissolved into each quart of ordinary tap water to make a bath sufficient deep to make prolonged contact with the desired part of the body. The affected skin needs to be soaked in this solution for at least 15 minutes at a time, preferably twice a day, air dried without rinsing. If callus is associated with the fungus, then a porous stone or coarse towel can be used after soaking, to remove loose tissue. For nail fungus, the affected parts should be soaked for 20 minutes, twice a day in the solution, then towelled dry thoroughly.

To be used as a douche for vaginal yeast infections: two tablespoons of my compound is mixed into a pint of tap water. The douche should be retained for five to ten minutes, then discarded, The area may be then rinsed with fresh water.

To prepare a treatment salve or ointment from my compound, a equal amount of my compound is mixed with petrolatum usp, by weight. The resulting ointment is applied to the affected area, as needed.

It has been found that application of the salve or ointment to the affected area after showering and drying is recommended. A thin layer of the prepared ointment should be applied to the affected area on the skin or nail then covered with a bandage, such as a “BANDAID” in order for the salve to be maintained in contact for appreciable periods of time. If the area becomes irritated, then the area should be flushed with tap water and dried. Later the area may be treated as before.

While the above description contains many specifications, they should not be construed as limitations on the scope of the invention, but rather as an exemplification of one preferred embodiment thereof. Many other variations are possible. For example, when compounding the resulting mixture of salts, additions of fillers, various coloring agents and stabilizers to the mixture are well known to person in the pertinent art. Accordingly, the scope of the invention should be determined not by the embodiment illustrated, but by the appended claims and their legal equivalents.

I claim:

1. A mixture for topical treatment of tinea cruris, persistent diabetic lesions, perianal rash, fungus and yeast infections comprising aquamarine salts having a chloride to sodium ratio of 1.4:1, iodized sodium chloride and sodium bicarbonate.

2. The mixture of claim 1, wherein the mixture comprises about three parts aquamarine salts, two parts iodized sodium chloride and one part sodium bicarbonate by weight.

3. The mixture of claim 1, wherein the mixture is dissolved in water.

4. The mixture of claim 1, wherein said mixture is ground into a fine powder.

5. The mixture of claim 4, wherein, petroleum gel is added.

6. The mixture of claim 1, wherein food coloring is added.

7. The mixture of claim 5, wherein food coloring is added.

8. the mixture of claim 3 wherein a ratio of four tablespoons of the mixture is dissolved into a liter of the water to form a bath.

9. The mixture of claim 3 wherein a ratio of two tablespoons of the mixture is dissolved into 16 ounces of water to prepare a vaginal douche.

10. The mixture of claim 5 wherein a ratio of petroleum gel to the mixture is 1:1, by weight.

11. The process of preparation of a mixture for topical treatment of tinea cruris, persistent diabetic lesions, perianal rash, fungus and yeast infections comprising: combining aquamarine salts having a chloride to sodium ratio of 1.4:1, iodized sodium chloride and sodium bicarbonate together.

12. The process of claim 11, wherein said combination is in the ratio of three parts aquamarine salts, two parts iodized sodium chloride and one part sodium bicarbonate, by weight.

13. The process of claim 11, wherein said process includes dissolution of said mixture in water to form a bath.

14. The process of claim 11, wherein said process includes the step of grinding said mixture into a fine powder for dry application.

15. The process of claim 14, wherein grinding step is followed by adding and then mixing petroleum gel into the powder.

16. The process of claim 11, wherein food coloring is added.

17. The process of claim 15, wherein food coloring is added.

18. The process of claim 13, wherein a ratio of four tablespoons of said combination is added to one liter of water to prepare a bath.

19. The process of claim 13, wherein a ratio of two tablespoons of said combination is added to 16 ounces of water to prepare a douche for vaginal application.

20. The process of claim 15, wherein the ratio of petroleum gel to powder is 1:1, by weight.
